SEQUENCE LISTING (12 sequences)

SEQ ID NO: 1 - DNA, 2030 bp, Homo sapiens
gcgcgatggcggcggctcctttaggcagctgaaaggggatttaggcccggaagatccgagtccatccgcggcggggagagggcaagcgggaccggtaggggccggagcagcggcggcggcgctcggactgtcccatccgccccgtattgaggcgctgggagcggcggggcgacaggaaagcgatggtgaaagcggggccgtgaggggggcggagccgggagccggacccgcagtagcggcagcagcggcgccgcctcccagagttcagacccaggaagcggccgggagggcaggagcgaatcgggccgccgccgccatggagctgagagtcgggaacaggtaccggctgggccggaagatcggcagcggctccttcggagacatctatctcggtacggacattgctgcaggagaagaggttgccatcaagcttgaatgtgtcaaaaccaaacaccctcagctccacattgagagcaaaatctacaagatgatgcagggaggagtgggcatccccaccatcagatggtgcggggcagagggggactacaacgtcatggtgatggagctgctggggccaagcctggaggacctcttcaacttctgctccaggaaattcagcctcaaaaccgtcctgctgcttgctgaccaaatgatcagtcgcatcgaatacattcattcaaagaacttcatccaccgggatgtgaagccagacaacttcctcatgggcctggggaagaagggcaacctggtgtacatcatcgacttcgggctggccaagaagtaccgggatgcacgcacccaccagcacatcccctatcgtgagaacaagaacctcacggggacggcgcggtacgcctccatcaacacgcaccttggaattgaacaatcccgaagagatgacttggagtctctgggctacgtgctaatgtacttcaacctgggctctctcccctggcaggggctgaaggctgccaccaagagacagaaatacgaaaggattagcgagaagaaaatgtccacccccatcgaagtgttgtgtaaaggctacccttccgaatttgccacatacctgaatttctgccgttccttgcgttttgacgacaagcctgactactcgtacctgcggcagcttttccggaatctgttccatcgccagggcttctcctatgactacgtgttcgactggaacatgctcaaatttggtgccagccgggccgccgatgacgccgagcgggagcgcagggaccgagaggagcggctgagacactcgcggaacccggctacccgcggcctcccttccacagcctccggccgcctgcgggggacgcaggaagtggctccccccacacccctcacccctacctcacacacggctaacacctccccccggcccgtctccggcatggagagagagcggaaagtgagtatgcggctgcaccgcggggcccccgtcaacatctcctcgtccgacctcacaggccgacaagatacctctcgcatgtccacctcacagattcctggtcgggtggcttccagtggtcttcagtctgtcgtgcaccgatgagaactctccttattgctgtgaagggcagacaatgcatggctgatctactctgttaccaatggctttactagtgacacgtcccccggtctaggatcgaaatgttaacaccgggagctctccaggccactcacccagcgacgctcgtgggggaaacatactaaacggacagactccaagagctgccaccgctggggctgcactgcggccccccacgtgaactcggttgtaacggggctgggaagaaaagcagagagagaattgcagagaatcagactccttttccagggcctcagctccctccagtggtggccgccctgtactccctgacgattccactgtaactaccaatcttctacttggttaagacagttttgtatcattttgctaaaaattattggcttaaatctgtgtaaagaaaaaaaaaaaaaaaaaaaaaaaaaaaaaaaaaaaaaaaaaaaaaaaaaaa

SEQ ID NO: 2 - DNA, 2106 bp, Homo sapiens
gcgcgatggcggcggctcctttaggcagctgaaaggggatttaggcccggaagatccgagtccatccgcggcggggagagggcaagcgggaccggtaggggccggagcagcggcggcggcgctcggactgtcccatccgccccgtattgaggcgctgggagcggcggggcgacaggaaagcgatggtgaaagcggggccgtgaggggggcggagccgggagccggacccgcagtagcggcagcagcggcgccgcctcccagagttcagacccaggaagcggccgggagggcaggagcgaatcgggccgccgccgccatggagctgagagtcgggaacaggtaccggctgggccggaagatcggcagcggctccttcggagacatctatctcggtacggacattgctgcaggagaagaggttgccatcaagcttgaatgtgtcaaaaccaaacaccctcagctccacattgagagcaaaatctacaagatgatgcagggaggagtgggcatccccaccatcagatggtgcggggcagagggggactacaacgtcatggtgatggagctgctggggccaagcctggaggacctcttcaacttctgctccaggaaattcagcctcaaaaccgtcctgctgcttgctgaccaaatgatcagtcgcatcgaatacattcattcaaagaacttcatccaccgggatgtgaagccagacaacttcctcatgggcctggggaagaagggcaacctggtgtacatcatcgacttcgggctggccaagaagtaccgggatgcacgcacccaccagcacatcccctatcgtgagaacaagaacctcacggggacggcgcggtacgcctccatcaacacgcaccttggaattgaacaatcccgaagagatgacttggagtctctgggctacgtgctaatgtacttcaacctgggctctctcccctggcaggggctgaaggctgccaccaagagacagaaatacgaaaggattagcgagaagaaaatgtccacccccatcgaagtgttgtgtaaaggctacccttccgaatttgccacatacctgaatttctgccgttccttgcgttttgacgacaagcctgactactcgtacctgcggcagcttttccggaatctgttccatcgccagggcttctcctatgactacgtgttcgactggaacatgctcaaatttggtgccagccgggccgccgatgacgccgagcgggagcgcagggaccgagaggagcggctgagacactcgcggaacccggctacccgcggcctcccttccacagcctccggccgcctgcgggggacgcaggaagtggctccccccacacccctcacccctacctcacacacggctaacacctccccccggcccgtctccggcatggagagagagcggaaagtgagtatgcggctgcaccgcggggcccccgtcaacatctcctcgtccgacctcacaggccgacaagatacctctcgcatgtccacctcacagaatagcattcctttcgaacaccacggcaagtagctgctcgtctcccatcggaaggcagcactggattcctggtcgggtggcttccagtggtcttcagtctgtcgtgcaccgatgagaactctccttattgctgtgaagggcagacaatgcatggctgatctactctgttaccaatggctttactagtgacacgtcccccggtctaggatcgaaatgttaacaccgggagctctccaggccactcacccagcgacgctcgtgggggaaacatactaaacggacagactccaagagctgccaccgctggggctgcactgcggccccccacgtgaactcggttgtaacggggctgggaagaaaagcagagagagaattgcagagaatcagactccttttccagggcctcagctccctccagtggtggccgccctgtactccctgacgattccactgtaactaccaatcttctacttggttaagacagttttgtatcattttgctaaaaattattggcttaaatctgtgtaaagaaaaaaaaaaaaaaaaaaaaaaaaaaaaaaaaaaaaaaaaaaaaaaaaaaaaaaaaaaaaaaa

SEQ ID NO: 3 - protein, 415 aa, Homo sapiens [sequence omitted]

SEQ ID NO: 4 - protein, 409 aa, Homo sapiens
MELRVGNRYRLGRKIGSGSFGDIYLGTDIAAGEEVAIKLECVKTKHPQLHIESKIYKMMQGGVGIPTIRWCGAEGDYNVMVMELLGPSLEDLFNFCSRKFSLKTVLLLADQMISRIEYIHSKNFIHRDVKPDNFLMGLGKKGNLVYIIDFGLAKKYRDARTHQHIPYRENKNLTGTARYASINTHLGIEQSRRDDLESLGYVLMYFNLGSLPWQGLKAATKRQKYERISEKKMSTPIEVLCKGYPSEFATYLNFCRSLRFDDKPDYSYLRQLFRNLFHRQGFSYDYVFDWNMLKFGASRAADDAERERRDREERLRHSRNPATRGLPSTASGRLRGTQEVAPPTPLTPTSHTANTSPRPVSGMERERKVSMRLHRGAPVNISSSDLTGRQDTSRMSTSQNSIPFEHHGK

SEQ ID NO: 5 - DNA, 2030 bp, Homo sapiens
gcgcgatggcggcggctcctttaggcagctgaaaggggatttaggcccggaagatccgagtccatccgcggcggggagagggcaagcgggaccggtaggggccggagcagcggcggcggcgctcggactgtcccatccgccccgtattgaggcgctgggagcggcggggcgacaggaaagcgatggtgaaagcggggccgtgaggggggcggagccgggagccggacccgcagtagcggcagcagcggcgccgcctcccagagttcagacccaggaagcggccgggagggcaggagcgaatcgggccgccgccgccatggagctgagagtcgggaacaggtaccggctgggccggaagatcggcagcggctccttcggagacatctatctcggtacggacattgctgcaggagaagaggttgccatcaagcttgaatgtgtcaaagccaaacaccctcagctccacattgagagcaaaatctacaagatgatgcagggaggagtgggcatccccaccatcagatggtgcggggcagagggggactacaacgtcatggtgatggagctgctggggccaagcctggaggacctcttcaacttctgctccaggaaattcagcctcaaaaccgtcctgctgcttgctgaccaaatgatcagtcgcatcgaatacattcattcaaagaacttcatccaccgggatgtgaagccagacaacttcctcatgggcctggggaagaagggcaacctggtgtacatcatcgacttcgggctggccaagaagtaccgggatgcacgcacccaccagcacatcccctatcgtgagaacaagaacctcacggggacggcgcggtacgcctccatcaacacgcaccttggaattgaacaatcccgaagagatgacttggagtctctgggctacgtgctaatgtacttcaacctgggctctctcccctggcaggggctgaaggctgccaccaagagacagaaatacgaaaggattagcgagaagaaaatgtccacccccatcgaagtgttgtgtaaaggctacccttccgaatttgccacatacctgaatttctgccgttccttgcgttttgacgacaagcctgactactcgtacctgcggcagcttttccggaatctgttccatcgccagggcttctcctatgactacgtgttcgactggaacatgctcaaatttggtgccagccgggccgccgatgacgccgagcgggagcgcagggaccgagaggagcggctgagacactcgcggaacccggctacccgcggcctcccttccacagcctccggccgcctgcgggggacgcaggaagtggctccccccacacccctcacccctacctcacacacggctaacacctccccccggcccgtctccggcatggagagagagcggaaagtgagtatgcggctgcaccgcggggcccccgtcaacatctcctcgtccgacctcacaggccgacaagatacctctcgcatgtccacctcacagattcctggtcgggtggcttccagtggtcttcagtctgtcgtgcaccgatgagaactctccttattgctgtgaagggcagacaatgcatggctgatctactctgttaccaatggctttactagtgacacgtcccccggtctaggatcgaaatgttaacaccgggagctctccaggccactcacccagcgacgctcgtgggggaaacatactaaacggacagactccaagagctgccaccgctggggctgcactgcggccccccacgtgaactcggttgtaacggggctgggaagaaaagcagagagagaattgcagagaatcagactccttttccagggcctcagctccctccagtggtggccgccctgtactccctgacgattccactgtaactaccaatcttctacttggttaagacagttttgtatcattttgctaaaaattattggcttaaatctgtgtaaagaaaaaaaaaaaaaaaaaaaaaaaaaaaaaaaaaaaaaaaaaaaaaaaaaaa

SEQ ID NO: 6 - DNA, 2106 bp, Homo sapiens
gcgcgatggcggcggctcctttaggcagctgaaaggggatttaggcccggaagatccgagtccatccgcggcggggagagggcaagcgggaccggtaggggccggagcagcggcggcggcgctcggactgtcccatccgccccgtattgaggcgctgggagcggcggggcgacaggaaagcgatggtgaaagcggggccgtgaggggggcggagccgggagccggacccgcagtagcggcagcagcggcgccgcctcccagagttcagacccaggaagcggccgggagggcaggagcgaatcgggccgccgccgccatggagctgagagtcgggaacaggtaccggctgggccggaagatcggcagcggctccttcggagacatctatctcggtacggacattgctgcaggagaagaggttgccatcaagcttgaatgtgtcaaagccaaacaccctcagctccacattgagagcaaaatctacaagatgatgcagggaggagtgggcatccccaccatcagatggtgcggggcagagggggactacaacgtcatggtgatggagctgctggggccaagcctggaggacctcttcaacttctgctccaggaaattcagcctcaaaaccgtcctgctgcttgctgaccaaatgatcagtcgcatcgaatacattcattcaaagaacttcatccaccgggatgtgaagccagacaacttcctcatgggcctggggaagaagggcaacctggtgtacatcatcgacttcgggctggccaagaagtaccgggatgcacgcacccaccagcacatcccctatcgtgagaacaagaacctcacggggacggcgcggtacgcctccatcaacacgcaccttggaattgaacaatcccgaagagatgacttggagtctctgggctacgtgctaatgtacttcaacctgggctctctcccctggcaggggctgaaggctgccaccaagagacagaaatacgaaaggattagcgagaagaaaatgtccacccccatcgaagtgttgtgtaaaggctacccttccgaatttgccacatacctgaatttctgccgttccttgcgttttgacgacaagcctgactactcgtacctgcggcagcttttccggaatctgttccatcgccagggcttctcctatgactacgtgttcgactggaacatgctcaaatttggtgccagccgggccgccgatgacgccgagcgggagcgcagggaccgagaggagcggctgagacactcgcggaacccggctacccgcggcctcccttccacagcctccggccgcctgcgggggacgcaggaagtggctccccccacacccctcacccctacctcacacacggctaacacctccccccggcccgtctccggcatggagagagagcggaaagtgagtatgcggctgcaccgcggggcccccgtcaacatctcctcgtccgacctcacaggccgacaagatacctctcgcatgtccacctcacagaatagcattcctttcgaacaccacggcaagtagctgctcgtctcccatcggaaggcagcactggattcctggtcgggtggcttccagtggtcttcagtctgtcgtgcaccgatgagaactctccttattgctgtgaagggcagacaatgcatggctgatctactctgttaccaatggctttactagtgacacgtcccccggtctaggatcgaaatgttaacaccgggagctctccaggccactcacccagcgacgctcgtgggggaaacatactaaacggacagactccaagagctgccaccgctggggctgcactgcggccccccacgtgaactcggttgtaacggggctgggaagaaaagcagagagagaattgcagagaatcagactccttttccagggcctcagctccctccagtggtggccgccctgtactccctgacgattccactgtaactaccaatcttctacttggttaagacagttttgtatcattttgctaaaaattattggcttaaatctgtgtaaagaaaaaaaaaaaaaaaaaaaaaaaaaaaaaaaaaaaaaaaaaaaaaaaaaaaaaaaaaaaaaaa

SEQ ID NO: 7 - protein, 415 aa, Homo sapiens
MELRVGNRYRLGRKIGSGSFGDIYLGTDIAAGEEVAIKLECVKAKHPQLHIESKIYKMMQGGVGIPTIRWCGAEGDYNVMVMELLGPSLEDLFNFCSRKFSLKTVLLLADQMISRIEYIHSKNFIHRDVKPDNFLMGLGKKGNLVYIIDFGLAKKYRDARTHQHIPYRENKNLTGTARYASINTHLGIEQSRRDDLESLGYVLMYFNLGSLPWQGLKAATKRQKYERISEKKMSTPIEVLCKGYPSEFATYLNFCRSLRFDDKPDYSYLRQLFRNLFHRQGFSYDYVFDWNMLKFGASRAADDAERERRDREERLRHSRNPATRGLPSTASGRLRGTQEVAPPTPLTPTSHTANTSPRPVSGMERERKVSMRLHRGAPVNISSSDLTGRQDTSRMSTSQIPGRVASSGLQSVVHR

SEQ ID NO: 8 - protein, 409 aa, Homo sapiens
MELRVGNRYRLGRKIGSGSFGDIYLGTDIAAGEEVAIKLECVKAKHPQLHIESKIYKMMQGGVGIPTIRWCGAEGDYNVMVMELLGPSLEDLFNFCSRKFSLKTVLLLADQMISRIEYIHSKNFIHRDVKPDNFLMGLGKKGNLVYIIDFGLAKKYRDARTHQHIPYRENKNLTGTARYASINTHLGIEQSRRDDLESLGYVLMYFNLGSLPWQGLKAATKRQKYERISEKKMSTPIEVLCKGYPSEFATYLNFCRSLRFDDKPDYSYLRQLFRNLFHRQGFSYDYVFDWNMLKFGASRAADDAERERRDREERLRHSRNPATRGLPSTASGRLRGTQEVAPPTPLTPTSHTANTSPRPVSGMERERKVSMRLHRGAPVNISSSDLTGRQDTSRMSTSQNSIPFEHHGK

SEQ ID NO: 9 - DNA, 1559 bp, Homo sapiens
gggaggcggcggcggcggcggcggcggcggcgagagcccagagccagagcccggccggggccgagcggagcgcggcggcggcggcggcggcggcggctgggccgggagaggctggcgcgccgggcggctccgcgaatcctccggcatccgccccggcgggccgcccccgcccgcggcagccccccgagcagtggcccggcatcggcgccttcccggcgggcaagagtgagccatggagctacgtgtggggaacaagtaccgcctgggacggaagatcgggagcgggtccttcggagatatctacctgggtgccaacatcgcctctggtgaggaagtcgccatcaagctggagtgtgtgaagacaaagcacccccagctgcacatcgagagcaagttctacaagatgatgcagggtggcgtggggatcccgtccatcaagtggtgcggagctgagggcgactacaacgtgatggtcatggagctgctggggcctagcctcgaggacctgttcaacttctgttcccgcaaattcagcctcaagacggtgctgctcttggccgaccagatgatcagccgcatcgagtatatccactccaagaacttcatccaccgggacgtcaagcccgacaacttcctcatggggctggggaagaagggcaacctggtctacatcatcgacttcggcctggccaagaagtaccgggacgcccgcacccaccagcacattccctaccgggaaaacaagaacctgaccggcacggcccgctacgcttccatcaacacgcacctgggcattgagcaaagccgtcgagatgacctggagagcctgggctacgtgctcatgtacttcaacctgggctccctgccctggcaggggctcaaagcagccaccaagcgccagaagtatgaacggatcagcgagaagaagatgtcaacgcccatcgaggtcctctgcaaaggctatccctccgaattctcaacatacctcaacttctgccgctccctgcggtttgacgacaagcccgactactcttacctacgtcagctcttccgcaacctcttccaccggcagggcttctcctatgactacgtctttgactggaacatgctgaaattcggtgcagcccggaatcccgaggatgtggaccgggagcggcgagaacacgaacgcgaggagaggatggggcagctacgggggtccgcgacccgagccctgccccctggcccacccacgggggccactgccaaccggctccgcagtgccgccgagcccgtggcttccacgccagcctcccgcatccagccggctggcaatacttctcccagagcgatctcgcgggtcgaccgggagaggaaggtgagtatgaggctgcacaggggtgcgcccgccaacgtctcctcctcagacctcactgggcggcaagaggtctcccggatcccagcctcacagacaagtgtgccatttgaccatctcgggaagtgaggagagcccccattggaccagtgtttgcttagtgtcttcactgtattttctttaaaaaaaaaaaaaaaaaaaaaaa

SEQ ID NO: 10 - protein, 416 aa, Homo sapiens
MELRVGNKYRLGRKIGSGSFGDIYLGANIASGEEVAIKLECVKTKHPQLHIESKFYKMMQGGVGIPSIKWCGAEGDYNVMVMELLGPSLEDLFNFCSRKFSLKTVLLLADQMISRIEYIHSKNFIHRDVKPDNFLMGLGKKGNLVYIIDFGLAKKYRDARTHQHIPYRENKNLTGTARYASINTHLGIEQSRRDDLESLGYVLMYFNLGSLPWQGLKAATKRQKYERISEKKMSTPIEVLCKGYPSEFSTYLNFCRSLRFDDKPDYSYLRQLFRNLFHRQGFSYDYVFDWNMLKFGAARNPEDVDRERREHEREERMGQLRGSATRALPPGPPTGATANRLRSAAEPVASTPASRIQPAGNTSPRAISRVDRERKVSMRLHRGAPANVSSSDLTGRQEVSRIPASQTSVPFDHLGK

SEQ ID NO: 11 - DNA, 1559 bp, Homo sapiens
gggaggcggcggcggcggcggcggcggcggcgagagcccagagccagagcccggccggggccgagcggagcgcggcggcggcggcggcggcggcggctgggccgggagaggctggcgcgccgggcggctccgcgaatcctccggcatccgccccggcgggccgcccccgcccgcggcagccccccgagcagtggcccggcatcggcgccttcccggcgggcaagagtgagccatggagctacgtgtggggaacaagtaccgcctgggacggaagatcgggagcgggtccttcggagatatctacctgggtgccaacatcacctctggtgaggaagtcgccatcaagctggagtgtgtgaagacaaagcacccccagctgcacatcgagagcaagttctacaagatgatgcagggtggcgtggggatcccgtccatcaagtggtgcggagctgagggcgactacaacgtgatggtcatggagctgctggggcctagcctcgaggacctgttcaacttctgttcccgcaaattcagcctcaagacggtgctgctcttggccgaccagatgatcagccgcatcgagtatatccactccaagaacttcatccaccgggacgtcaagcccgacaacttcctcatggggctggggaagaagggcaacctggtctacatcatcgacttcggcctggccaagaagtaccgggacgcccgcacccaccagcacattccctaccgggaaaacaagaacctgaccggcacggcccgctacgcttccatcaacacgcacctgggcattgagcaaagccgtcgagatgacctggagagcctgggctacgtgctcatgtacttcaacctgggctccctgccctggcaggggctcaaagcagccaccaagcgccagaagtatgaacggatcagcgagaagaagatgtcaacgcccatcgaggtcctctgcaaaggctatccctccgaattctcaacatacctcaacttctgccgctccctgcggtttgacgacaagcccgactactcttacctacgtcagctcttccgcaacctcttccaccggcagggcttctcctatgactacgtctttgactggaacatgctgaaattcggtgcagcccggaatcccgaggatgtggaccgggagcggcgagaacacgaacgcgaggagaggatggggcagctacgggggtccgcgacccgagccctgccccctggcccacccacgggggccactgccaaccggctccgcagtgccgccgagcccgtggcttccacgccagcctcccgcatccagccggctggcaatacttctcccagagcgatctcgcgggtcgaccgggagaggaaggtgagtatgaggctgcacaggggtgcgcccgccaacgtctcctcctcagacctcactgggcggcaagaggtctcccggatcccagcctcacagacaagtgtgccatttgaccatctcgggaagtgaggagagcccccattggaccagtgtttgcttagtgtcttcactgtattttctttaaaaaaaaaaaaaaaaaaaaaaa

SEQ ID NO: 12 - protein, 416 aa, Homo sapiens
MELRVGNKYRLGRKIGSGSFGDIYLGANITSGEEVAIKLECVKTKHPQLHIESKFYKMMQGGVGIPSIKWCGAEGDYNVMVMELLGPSLEDLFNFCSRKFSLKTVLLLADQMISRIEYIHSKNFIHRDVKPDNFLMGLGKKGNLVYIIDFGLAKKYRDARTHQHIPYRENKNLTGTARYASINTHLGIEQSRRDDLESLGYVLMYFNLGSLPWQGLKAATKRQKYERISEKKMSTPIEVLCKGYPSEFSTYLNFCRSLRFDDKPDYSYLRQLFRNLFHRQGFSYDYVFDWNMLKFGAARNPEDVDRERREHEREERMGQLRGSATRALPPGPPTGATANRLRSAAEPVASTPASRIQPAGNTSPRAISRVDRERKVSMRLHRGAPANVSSSDLTGRQEVSRIPASQTSVPFDHLGK

(19) United States
(12) Patent Application Publication
(10) Pub. No.: US 2006/0134619 A1
(43) Pub. Date: Jun. 22, 2006

(54) Casein kinase i epsilon and casein kinase i delta and sleep in humans

(76) Inventors: Louis Ptacek, San Francisco, CA (US); Christopher Jones, Salt Lake City, UT (US); Ying Hui Fu, San Francisco, CA (US)

Correspondence Address: MADSON & AUSTIN;GATEWAY TOWER WEST, SUITE 900, 15 WEST SOUTH TEMPLE, SALT LAKE CITY, UT, 84101, US

(21) Appl. No.: 10/536,735

(22) PCT Filed: Nov. 26, 2003
(86) PCT No.: PCT/US03/37992

§ 371 Date: Nov. 17, 2005

Related U.S. Application Data
(60) Provisional application No. 60/429,930, filed on Nov. 27, 2002.

(51) Int. Cl. C12Q 1/68; C07H 21/04; C12P 21/06; C12N 9/12

(52) U.S. Cl. 435/6; 435/69.1; 435/194; 435/320.1; 435/325; 536/23.2

(57) ABSTRACT

Genetic variants of the casein kinase I delta and casein kinase I epsilon genes are disclosed. Herein, two novel mutant casein kinase I delta genes and a novel mutant casein kinase I epsilon gene are disclosed. The protein products of the mutant casein kinase I delta gene and the mutant casein kinase I epsilon gene are also disclosed. The nucleic acid sequences of the invention lead to familial advanced sleep phase syndrome.

### RELATED APPLICATIONS

This application is related to and claims the benefit of U.S. Provisional Patent Application No. 60/429,930 filed Nov. 27, 2002, and entitled “Casein Kinase I Epsilon and Casein Kinase I Delta and Sleep in Humans,” which is incorporated herein by reference.

### STATEMENT REGARDING FEDERALLY SPONSORED RESEARCH

This invention was made with government support under Grant Number HL59596 awarded by the United States Department of Health and Human Services of the National Institutes of Health. The Government has certain rights to this invention.

### BACKGROUND OF THE INVENTION

The International Classification of Sleep Disorders lists approximately 60 disorders of human sleep. Association, A.S.D., International classification of sleep disorders: Diagnostic and coding manual, 1997, Rochester. The main categories of sleep-wake complaint in clinical practice are excessive daytime sleepiness (“EDS”), difficulty initiating and/or maintaining sleep (“DIMS”); and unwanted behaviors arising out of sleep. The most common of these sleep disorders are obstructive sleep apnea (with EDS), anxious and depressive features (with DIMS), restless legs syndrome (with DIMS and/or EDS), narcolepsy (with EDS), and the circadian (i.e. daily sleep schedule) disorders of either delayed or advanced sleep phase syndromes (“DSPS” or “ASPS”). Circadian sleep schedule disorders are common in young and elderly patients alike, and often cause significant sleep deprivation. The behavioral, cognitive and memory impairments caused by sleep deprivation have been shown to adversely affect driving and work safety, social function, school performance, and overall quality of life.

The master circadian pacemaker in mammals is located in the suprachiasmatic nucleus (SCN) of the anterior hypothalamus. Ibuka & Kawamura, Loss of circadian rhythm in sleep-wakefulness cycle in the rat by suprachiasmatic nucleus lesions, Brain Res., 1975, 96(1):76-81. The SCN rhythms of firing rate and gene expression, and thus the sleep-wake and other bodily rhythms are entrained to the 24-hour solar day primarily via photic information. This information is most likely transduced by retinal ambient light receptors in which melanopsin may play a role. Czeisler, C. A., et al., Bright light induction of strong (type 0) resetting of the human circadian pacemaker, Science, 1989, 244(4910):1328-33; Moore, R. Y., Retinohypothalamic projection in mammals: a comparative study, Brain Res., 1973, 49(2):403-9.

It has been observed that, in most cases of advanced phase sleep syndrome, or “ASPS,” and delayed sleep phase syndrome, or “DSPS,” the entire sleep-wake cycle is shifted either earlier or later, respectively, with respect to solar time. The phenomenon of “internal desynchronization” of the sleep-wake rhythm from melatonin or temperature rhythms connected with the circadian rhythm has led to the notion that the former is less tightly coupled to the SCN rhythm than the latter, thus making it necessary to measure the phase of both the sleep-wake and the melatonin or temperature rhythms to more fully describe how the circadian system is functioning. Wever, R. A., The circadian system in man, results of experiments under temporal isolation, 1979, Heidelberg: Springer-Verlag.

Individuals affected by the sleep phase disorders noted above are characterized by several traits. DSPS patients feel wide awake, energetic and motivated until late in the night. As a result and depending on the severity, sleep onset may be delayed until 1:00 a.m. to 6:00 a.m., and the circadian “morning” increase in alertness does not occur until approximately 10:00 a.m. to 2:00 p.m. Sleep phase-delayed individuals are often sleep deprived because sleep onset is delayed by the biological clock and morning wake up time is enforced by the alarm clock and social responsibilities. The prevalence of DSPS in the general population is thought to be high, especially in adolescents and young adults, but the precise prevalence is not known. There is currently much discussion in school districts across the country about whether school start times should be delayed for adolescents in order to increase their nightly sleep time and thus their academic and social performance. Foundation, N. S., Adolescent sleep needs and patterns, 2000, National Sleep Foundation: Washington, D.C.

People with ASPS fall asleep during what would be the “Maintenance of Wakefulness Zone” for conventional sleepers and tend to wake up alert and energetic in the early morning hours when most people are the sleepiest. ASPS patients are often presented with the difficulties both of staying awake to satisfy domestic responsibilities in the evening and of an obligate early morning awakening before other people are active. This can result in significant sleep deprivation if social responsibilities keep the patient awake late and their biological clock wakes them up early. Some people with ASPS sleep on their “biological” schedule, do not complain, and find that they can accomplish a great deal in the early morning without other people interrupting them. Therefore, ASPS may be seen as a condition or trait, and not always as a disabling “disorder.”

The most common cause of ASPS is the natural aging process, which is also associated with phase advance of the temperature rhythm. Czeisler, C. A., et al., Association of sleep-wake habits in older people with changes in output of circadian pacemaker, Lancet, 1992, 340(8825):933-6. The prevalence of ASPS in the elderly is high, but the precise prevalence is not known. The pathophysiology is also unknown, but recent evidence suggests that a shorter endogenous circadian period length, tau (τ), i.e. a “faster clock,” is not the explanation. Czeisler, C. A., et al., Stability, precision, and near-24-hour period of the human circadian pacemaker, Science, 1999, 284(5423):2177-81.

The spectrum of sleep schedule preference in the normal and younger population also includes many people with a modest “morning lark” tendency. Weak polygenic influences are suspected to be a cause of this characteristic based in part on heritability studies in twins and on candidate gene polymorphism correlations in large populations of apparently normal sleepers. Selby, J., et al., Morningness/eveningness is heritable, Society for Neuroscience Abstracts, 1992, 18:196; Katzenberg, D., et al., A CLOCK polymorphism associated with human diurnal preference, Sleep, 1998, 21(6):569-76. Autosomal dominant ASPS with profound sleep phase advance has been documented, but appears to be uncommon. Jones, C. R., et al., Familial advanced sleep-phase syndrome: A short-period circadian rhythm variant in humans, Nat. Med., 1999, 5(9):1062-5. In one subject, a remarkably short τ was the apparent explanation for the phase advance. There are only isolated case reports of post-traumatic ASPS. Govindan, S. and E. Govindan, Brain imaging in post traumatic circadian rhythm sleep disorders, Sleep Research, 1995, 24:A308.

Circadian dysrhythmias other than DSPS and ASPS include the non-24 hour sleep-wake disorder and imposed perturbations such as shift work schedules and “jet lag”. A non-24 hour sleep-wake schedule is seen in approximately 50% of people with complete retinal blindness. Sack, R. L., et al., Entrainment of free-running circadian rhythms by melatonin in blind people, N. Engl. J. Med., 2000, 343(15):1070-7. By some estimates, up to 20% of the work force is on some form of shift work schedule. Mellor, E. F., Shift work and flexitime: how prevalent are they?, in Monthly Labor Review, 1986, pp. 14-21.

The correlations between genotype and different aspects of circadian phenotype in different genetic causes of familial advanced sleep phase syndrome, or “FASPS” that would help elucidate molecular circadian mechanisms are either unknown or poorly described. For example, the variability in phase advance within one kindred with highly penetrant monogenic FASPS was shown by the inventors to be considerable, thus suggesting that polymorphisms in other candidate genes and/or environmental factors also influence the magnitude of phase advance. Jones, C. R., et al., Familial advanced sleep-phase syndrome: A short-period circadian rhythm variant in humans, Nat. Med., 1999. 5(9):1062-5. Whether such variability will be seen in other FASPS kindreds is unknown. It would also be of interest to compare the average severity of phase advance produced by different human ASPS mutations since currently only one mutation is known. Preliminary data gathered by the inventors demonstrated significantly more phase advance after just one day of imposed early evening dim light in FASPS subjects than controls. Id. Differences in this tendency for rapid phase advance could shed light on the how the formal properties of the clock are affected by different mutations. Three subtypes of ASPS based on differences in the phase angle of entrainment of the sleep-wake rhythm relative to the melatonin rhythm were predicted 10 years ago. Limited abstract and unpublished data lend support to two of these subtypes. Id., Lewy, A. J., Chronobiologic disorders, social cues, and the light-dark cycle, Chronobiol. Int., 1990, 7(1):15-21; Lewy, A. J., et al., Later circadian phase of plasma melatonin relative to usual waketime in older subjects, Sleep, 2000, 23:A188 (data not shown).

Additional descriptions of qualitatively different sleep vs. melatonin phase relationships in different ASPS mutations could therefore add new subtypes to the nosology of circadian dysrhythmias. Computer simulations and limited empirical human data support a relationship between a shorter endogenous τ and an earlier phase angle of entrainment of the sleep-wake and melatonin rhythms relative to the light-dark cycle. Klerman, E. B., et al., Simulations of light effects on the human circadian pacemaker: implications for assessment of intrinsic period, Am. J. Physiol., 1996, 270(1 Pt 2):R271-82; Sack, R. L., R. W. Brandes, and A. J. Lewy, Correlation of intrinsic circadian period with morningness-eveningness in young men, Sleep, 1999, 22:S92; Duffy, J., et al., Correlation of intrinsic circadian period with morningness-eveningness in young men, Sleep, 1999, 22(Suppl 1):S92.

Describing this relationship in people has been hampered by the relatively small range of τ among normal volunteers. The availability of human FASPS mutants would help overcome this limitation. It is currently not known how different human FASPS mutations might interact with the common trend toward phase delay during adolescence and phase advance during the geriatric years. A description of whether some FASPS mutations seem to be clinically silent during adolescence, or have striking progression of phase advance with age beyond the fifth decade might generate hypotheses on the molecular mechanisms of these common ontogenetic/age-related changes in circadian organization.

Within the last few years, there has been an explosion of new data regarding clock genes and mechanisms in a variety of organisms. Reppert, S. M., A clockwork explosion! Neuron, 1998, 21(1):1-4, Wager-Smith, K. and S. A. Kay, Circadian rhythm genetics: from flies to mice to humans, Nat. Genet., 2000, 26(1):23-7. Several proteins have been identified to be central to the design of the clock. In Drosophila, these include PER and TIM which act to repress transcription of their own genes in a negative feedback loop. They intermittently engage and disengage from transcriptional activators (CLK, CYC or BMAL) to form a dynamic multiprotein complex. Lee, C., K. Bae, and I. Edery, PER and TIM inhibit the DNA binding activity of a Drosophila CLOCK-CYC/dBMAL1 heterodimer without disrupting formation of the heterodimer: a basis for circadian transcription, Mol. Cell Diol., 1999, 19(8):5316-25. The “lag” produced between the transcriptional induction of per and tim and the nuclear translocation of the repressor proteins they encode creates a temporal separation between phases of induction and repression. This temporal separation therefore generates the important feature in the clock mechanism: oscillation. Dunlap, J. C., Molecular bases for circadian clocks, Cell, 1999, 96(2):271-90.

Among all species that have been studied, the Drosophila clock is best understood. Scully, A. L. and S. A. Kay, Time flies for Drosophila, Cell, 2000, 100(3):297-300. At around noon, the CLK protein together with its partner, CYC, bind to E-box DNA elements and activate a slow transcriptional induction of the per and tim genes. Lee, C., K. Bae, and I. Edery, PER and TIM inhibit the DNA binding activity of a Drosophila CLOCK-CYC/dBMAL1 heterodimer without disrupting formation of the heterodimer: a basis for circadian transcription, Mol. Cell Biol., 1999, 19(8):5316-25; Hao, H., D. L. Allen, and P. E. Hardin, A circadian enhancer mediates PER-dependent mRNA cycling in Drosophila melanogaster, Mol. Cell Biol., 1997, 17(7):3687-93, and Rutila, J. E., et al., CYCLE is a second bHLH-PAS clock protein essential for circadian rhythmicity and transcription of Drosophila period and timeless, Cell, 1998, 93(5):805-14. Per and tim RNA levels begin to rise, but DBT (a constitutively produced protein homologous to casein kinase I epsilon) reduces the stability (and thus the level of accumulation) of monomeric PER protein by phosphorylation. Price, J. L., et al., double-time is a novel Drosophila clock gene that regulates Period protein accumulation, Cell, 1998, 94(1):83-95. Nightfall allows TIM, a light sensitive protein, to rise to a level at which it can bind and protect PER protein from degradation and stable TIM:PER heterodimers begin to form. Id., Kloss, B., et al., The Drosophila clock gene double-time encodes a protein closely related to human casein kinase I epsilon, Cell, 1998, 94(1):97-107.

By midnight, TIM:PER heterodimers have translocated into the nucleus and have physically associated with CLK:CYC complexes. Young, M. W., The molecular control of circadian behavioral rhythms and their entrainment in Drosophila, Annu. Rev. Biochem., 1998, 67:135-52. This association inhibits the ability of the CLK:CYC protein complex to bind DNA and therefore transcription of these genes ceases. Darlington, T. K., et al., Closing the circadian loop: CLOCK-induced transcription of its own inhibitors per and tim, Science, 1998, 280(5369):1599-603; and Lee, C., K. Bae, and I. Edery, The Drosophila CLOCK protein undergoes daily rhythms in abundance, phosphorylation, and interactions with the PER-TIM complex, Neuron, 1998, 21(4):857-67. The mRNA levels of per and tim then decline throughout the night. Daybreak stimulates the photoreceptor, CRY to sequester TIM protein and diminish its function as a transcriptional regulator. Emery, P., et al., CRY, a Drosophila clock and light-regulated cryptochrome, is a major contributor to circadian rhythm resetting and photosensitivity, Cell, 1998, 95(5):669-79; Stanewsky, R., et al., The cryb mutation identifies cryptochrome as a circadian photoreceptor in Drosophila, Cell, 1998, 95(5):681-92. TIM becomes phosphorylated, ubiquitinated and degraded via the proteasomal pathway by the induction of light. Naidoo, N., et al., A role for the proteasome in the light response of the timeless clock protein, Science, 1999, 285(5434):1737-41. By noon the second day, the levels of PER and TIM have decreased to where they can no longer inhibit CLK:CYC transcription activity and a new cycle of synthesis begins. This self-sustaining loop can be reset by the major entraining cue: light, which causes rapid TIM protein degradation.

Mammalian clock organization shares some similarities and differences with that of the fly. Homologues of the Drosophila circadian clock genes have been identified in mammals including: Clk, casein kinase I epsilon (“CkIε” a homolog of dbt), Cry1, Cry2, Per1, Per2, Per3, Bma1 (homologous to cyc), and Tim. Shearman, L. P., et al., Interacting molecular loops in the mammalian circadian clock, Science, 2000, 288(5468):1013-9. As in the fly, mammalian CLOCK and BMAL act as transcriptional activators on E-boxes found in mPer and other circadianly regulated promoters. PER negatively regulates the transcriptional activity of CLK and BMAL as in the fly. Dunlap, J. C., Molecular bases for circadian clocks, Cell, 1999, 96(2):271-90.

DBT (homologous to CKIε) phosphorylates and destabilizes PER in mammals as in flies. Keesler, G. A., et al., Phosphorylation and destabilization of human period I clock protein by human casein kinase I epsilon, Neuroreport, 2000, 11(5):951-5. However, several clock genes that are unique in the fly have multiple homologous copies in the mammalian genome. King, D. P. and J. S. Takahashi, Molecular genetics of circadian rhythms in mammals, Annu. Rev. Neurosci., 2000, 23:713-42. The physical interactions of some of the pacemaker proteins in fly have been found in mouse, but other interactions are specific to one or the other species.

Although the Drosophila and mouse circadian feedback loops have similar components, they function at opposite phases of the circadian cycle and mediate light-dependent phase resetting through different mechanisms. In Drosophila, transcription of the per and tim genes is activated by CLK:CYC late in the day and inhibited by PER and TIM late at night. In contrast, transcription of mPer1, 2, and 3 is activated by CLK:BMAL early in the day and repressed by Cry late in the day. Dunlap, J. C., Molecular bases for circadian clocks, Cell, 1999, 96(2):271-90. Despite these phase differences, Drosophila and mice show similar responses to light pulses administered during the dark phase. The mechanisms by which light resets the clock are very different in fly and mouse. In Drosophila, light leads to the degradation of TIM protein. dCRY acts as a circadian photoreceptor, resetting the clock through light-dependent interactions with TIM. Hunter Ensor, M., A. Ousley, and A. Sehgal, Regulation of the Drosophila protein timeless suggests a mechanism for resetting the circadian clock by light, Cell, 1996, 84(5):677-85; Lee, C., et al., Resetting the Drosophila clock by photic regulation of PER and a PER-TIM complex, Science, 1996, 271(5256):1740-4; Myers, M. P., et al., Light-induced degradation of TIMELESS and entrainment of the Drosophila circadian clock, Science, 1996, 271(5256):1736-40; and Zeng, H., et al., A light-entrainment mechanism for the Drosophila circadian clock, Nature, 1996, 380(6570):129-35.

Familial ASPS represents the first description of a monogenic circadian rhythm disorder in humans. Since ASPS is clearly a single gene Mendelian trait in the families studied, a very focused and directed effort at molecular characterization has an extremely high likelihood of success. Thus, it will provide a window into better understanding of human sleep physiology. The ubiquitous prevalence of circadian rhythms in nature—from primitive unicellular flagellates to insects, mammals, and primates—as well as the adaptive significance of circadian rhythms, suggests the possibility that clock mechanisms are evolutionarily conserved. In light of this, those differences discovered in comparative research may shed light on the genetic origins of behavioral differences such as nocturnality. This hypothesis can be tested as more is known about the molecular basis of human circadian physiology and will complement understanding of circadian rhythms studied in other animals. Molecular characterization of ASPS will lead to better understanding of normal sleep physiology and possibly to a better understanding of other human circadian sleep disorders including geriatric sleep phase advance, the sleep phase delay of adolescence and young adulthood, free-running rhythms of the blind, seasonal affective disease, and other forms of insomnia. This work may also have implications for sleep alterations in these settings.

In summary, despite the fact that the field of circadian rhythm genetics and biology has grown tremendously over the last decade, much of human circadian rhythm genetics is not well understood. Extensive additional study of human circadian rhythm mutations is required to further understand the similarities and difference between human clocks and those of other organisms. It would thus be an advancement in the art to disclose human gene variants which participate in circadian rhythm cycles in humans. More specifically, it would be an advancement in the art to provide human gene variants which participate in familial advanced sleep phase syndrome. Such gene variants are disclosed herein.

### BRIEF SUMMARY OF THE INVENTION

The present invention has been developed in response to the present state of the art, and in particular, in response to the problems and needs in the art that have not yet been fully solved by currently known genes and currently available sleep disorder therapies. The present invention provides mutants of the human casein kinase 1 delta gene and a mutant of the human casein kinase 1 epsilon gene which are each implicated in familial advanced sleep phase syndrome (“FASPS”).

The mutant gene sequences of the invention were identified in individuals suffering from FASPS and in some cases, individuals with a family history of FASPS. The sequences were checked against 200 control sets of DNA (having a total of 400 chromosomes) for mutations in casein kinase I delta isoform one, casein kinase I delta isoform two, and casein kinase I epsilon. The mutant casein kinase I delta and epsilon sequences of the invention were not identified in any of the control DNA sets. Subsequent studies of the families having the identified variants showed that the mutation of casein kinase I delta cosegregates in the family. The casein kinase I epsilon mutation occurs in a man whose father is said to have been affected, but who is now deceased.

Due to their involvement in human circadian rhythms, these genes and the proteins that they encode are excellent targets for the development of therapeutic agents that could alter human sleep and circadian rhythm patterns. Because the mutant casein kinase I delta and epsilon polypeptides expressed by the mutant polynucleotide sequences are enzymatic in nature, they are likely to be found to be susceptible to manipulation using chemical compounds that can increase or decrease their activity. As a result, the identification of genetic variants in casein kinase I epsilon and casein kinase I delta, when viewed with the implication of these genes in familial advanced sleep phase syndrome provides the rationale for developing such agents and using them to try to modulate human sleep.

Familial advanced sleep phase syndrome is a rare condition, but advanced sleep phase syndrome is very common as a part of normal aging. In fact, approximately one-third of individuals over age 65 have advanced sleep phase syndrome. This more common sleep disorder may also benefit from therapies derived for use with the genetically-based familial syndrome discussed above. In addition, other common sleep disorders might benefit from development of new FASPS-related therapies. Such disorders include delayed sleep phase syndrome (an epidemic in adolescents), as well as sleep disorders associated with jet lag, shift work, and other similar conditions.

In accordance with the invention as embodied and broadly described herein, mutants of the casein kinase I delta gene (SEQ ID NOS: 5, 6) and casein kinase I epsilon gene (SEQ ID NO: 11) are provided. The invention further provides polypeptides corresponding with these mutant genes.

One first such polynucleotide of the invention is a polynucleotide coding for mutant casein kinase I delta isoform one, the sequence of which is shown in SEQ ID NO: 7. Such polynucleotides include isolated nucleic acids coding for this mutant casein kinase I delta isoform, including mRNA, cDNA, and fragments thereof. One such polynucleotide coding for mutant casein kinase I delta isoform one has the sequence shown in SEQ ID NO: 5. The polynucleotides of the invention include degenerate polynucleotides coding for the sequence of mutant casein kinase I delta isoform one shown in SEQ ID NO: 7. The invention also provides polynucleotide sequences complementary to sequences encoding mutant casein kinase I delta isoform one, including polynucleotides complementary to degenerate polynucleotide sequences encoding mutant casein kinase I delta isoform one.

The invention further includes fragments of the polynucleotides described above. More specifically, the invention provides fragments useful in biological research applications, diagnostic applications, clinical applications and therapeutic applications which include at least 15 contiguous nucleotides from the polynucleotide of the polynucleotides listed above, each fragment including nucleotide 446 of the polynucleotide. Polynucleotide fragments include fragments of SEQ ID NO: 5 including nucleotide 446 of SEQ ID NO: 5.

The invention further provides vectors comprising the DNA polynucleotides of the invention. The invention also encompasses host cells including such vectors. Further, the invention includes methods of producing the polypeptides of the invention comprising the steps of allowing the host cells of the invention to express the polypeptide encoded by the polynucleotide. Similarly, the invention encompasses methods of producing cells expressing a polypeptide comprising the steps of transforming or transfecting cells with the vectors of the invention and allowing the cells to express the polypeptide encoded by the polynucleotide of the vector.

A next group of polynucleotides of the invention include polynucleotides coding for mutant casein kinase I delta isoform two, the sequence of which is shown in SEQ ID NO: 8. Such polynucleotides include isolated nucleic acids coding for this mutant casein kinase I delta isoform, including mRNA, cDNA, and fragments thereof. One such polynucleotide coding for mutant casein kinase I delta isoform two has the sequence shown in SEQ ID NO: 6. The polynucleotides of the invention include degenerate polynucleotides coding for the sequence of mutant casein kinase I delta isoform two shown in SEQ ID NO: 8. The invention also provides polynucleotide sequences complementary to sequences encoding mutant casein kinase I delta isoform two, including polynucleotides complementary to degenerate polynucleotide sequences encoding mutant casein kinase I delta isoform two.

The invention further includes fragments of the polynucleotides described above. More specifically, the invention provides fragments useful in biological research applications, diagnostic applications, clinical applications and therapeutic applications which include at least 15 contiguous nucleotides from the polynucleotide of the polynucleotides listed above, each fragment including nucleotide 446 of the polynucleotide. Polynucleotide fragments include fragments of SEQ ID NO: 6 including nucleotide 446 of SEQ ID NO: 6.

The invention further provides vectors comprising the DNA polynucleotides of the invention. The invention also encompasses host cells including such vectors. Further, the invention includes methods of producing the polypeptides of the invention comprising the steps of allowing the host cells of the invention to express the polypeptide encoded by the polynucleotide. Similarly, the invention encompasses methods of producing cells expressing a polypeptide comprising the steps of transforming or transfecting cells with the vectors of the invention and allowing the cells to express the polypeptide encoded by the polynucleotide of the vector.

A final group of polynucleotides of the invention include polynucleotides coding for mutant casein kinase I epsilon, the sequence of which is shown in SEQ ID NO: 12. Such polynucleotides include isolated nucleic acids coding for this mutant casein kinase I epsilon, including mRNA, cDNA, and fragments thereof. One such polynucleotide coding for mutant casein kinase I epsilon has the sequence shown in SEQ ID NO: 11. The polynucleotides of the invention include degenerate polynucleotides coding for the sequence of mutant casein kinase I epsilon shown in SEQ ID NO: 12. The invention also provides polynucleotide sequences complementary to sequences encoding mutant casein kinase I epsilon, including polynucleotides complementary to degenerate polynucleotide sequences encoding mutant casein kinase I epsilon.

The invention further includes fragments of the polynucleotides described above. More specifically, the invention provides fragments useful in biological research applications, diagnostic applications, clinical applications and therapeutic applications which include at least 15 contiguous nucleotides from the polynucleotide of the polynucleotides listed above, each fragment including nucleotide 320 of the polynucleotide. Polynucleotide fragments include fragments of SEQ ID NO: 11 including nucleotide 320 of SEQ ID NO: 11.

The invention further provides vectors comprising the DNA polynucleotides of the invention. The invention also encompasses host cells including such vectors. Further, the invention includes methods of producing the polypeptides of the invention comprising the steps of allowing the host cells of the invention to express the polypeptide encoded by the polynucleotide. Similarly, the invention encompasses methods of producing cells expressing a polypeptide comprising the steps of transforming or transfecting cells with the vectors of the invention and allowing the cells to express the polypeptide encoded by the polynucleotide of the vector.

The invention also provides methods of screening for potential agents that regulate the mutants of casein kinase I epsilon and casein kinase I delta identified in this application. According to these methods of the invention, a cell expressing or overexpressing the polypeptide of SEQ ID NOS: 7, 8, or 12, or a fragment thereof is contacted with a test compound. Following this contact, the activity of the polypeptide of SEQ ID NOS: 7, 8, or 12 is measured. Those test compounds that either increase or decrease the activity of the mutants of casein kinase I epsilon and casein kinase I delta of the invention are potential agents that regulate the activity of the mutant polypeptides. In those cells in which a fragment of SEQ ID NOS: 7 and 8 is expressed, according to the methods of the invention, the fragment would include position 44 of the mutant polypeptide. In those cells in which a fragment of SEQ ID NO: 12 is expressed, according to the methods of the invention, the fragment would include position 30 of the mutant polypeptide.

These and other features and advantages of the present invention will become more fully apparent from the following description and appended claims, or may be learned by the practice of the invention as set forth hereinafter.

### BRIEF DESCRIPTION OF THE FIGURES

A more particular description of the invention briefly described above will be rendered by reference to the appended figures. These figures only provide information concerning typical embodiments of the invention and are not therefore to be considered limiting of its scope.

FIG. 1 shows the sequence of the wild-type human casein kinase I delta isoform I cDNA (SEQ ID NO: 1);

FIG. 2 shows the sequence of the wild-type human casein kinase I delta isoform 2 cDNA (SEQ ID NO: 2);

FIG. 3A shows the amino acid sequence of the wild-type human casein kinase I delta isoform I (SEQ ID NO: 3);

FIG. 3B shows the amino acid sequence of the wild-type human casein kinase I delta isoform 2 (SEQ ID NO: 4);

FIG. 4 shows the sequence of the mutant human casein kinase I delta isoform 1 of the invention (SEQ ID NO: 5);

FIG. 5 shows the sequence of the mutant human casein kinase I delta isoform 2 of the invention (SEQ ID NO: 6);

FIG. 6A shows the amino acid sequence of the mutant human casein kinase I delta isoform 1 (SEQ ID NO: 7);

FIG. 6B shows the amino acid sequence of the mutant human casein kinase I delta isoform 2 (SEQ ID NO: 8);

FIG. 7 shows the sequence of the wild-type human casein kinase I epsilon cDNA (SEQ ID NO: 9);

FIG. 8 shows the amino acid sequence of the wild-type human casein kinase I epsilon (SEQ ID NO: 10);

FIG. 9 shows the sequence of the mutant human casein kinase I epsilon cDNA (SEQ ID NO: 11); and

FIG. 10 shows the amino acid sequence of the mutant human casein kinase I epsilon (SEQ ID NO: 12).

### DETAILED DESCRIPTION OF THE INVENTION

The practice of the present invention will employ, unless otherwise indicated, conventional methods of virology, microbiology, molecular biology, and recombinant DNA techniques within the skill of the art. Such techniques are fully explained in the literature. See, e.g., Sambrook, et al., Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields & D. M. Knipe, eds.).

All publications, patents and patent applications cited herein, whether supra or infra, are hereby incorporated by reference in their entirety. As used in this specification and the appended claims, the singular forms “a,” “and,” and “the” include plural references unless the content clearly dictates otherwise.

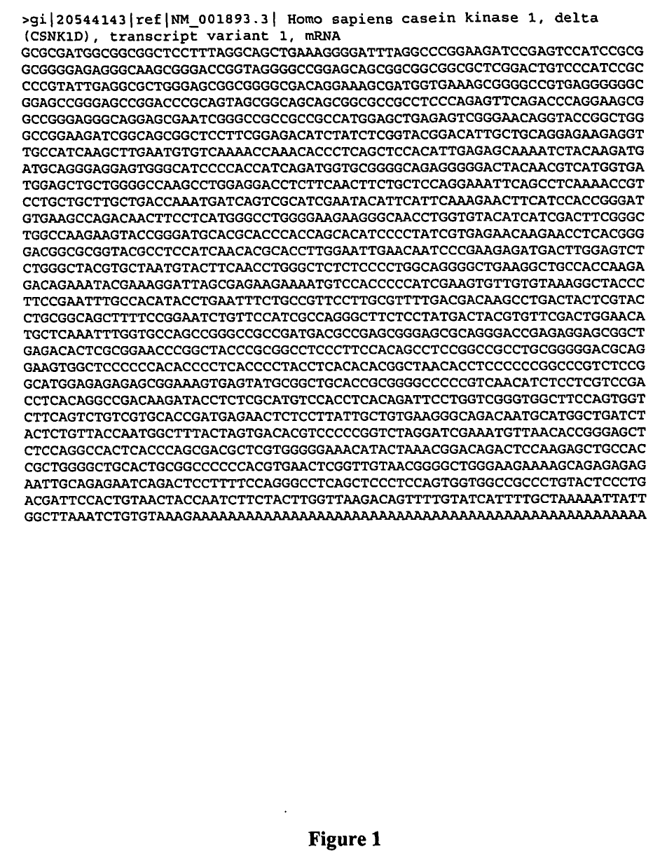

The present invention will be best understood by reference to the drawings and description herein. Thus, the following more detailed description of the embodiments of the present invention, as represented in FIGS. 1 through 10, is not intended to limit the scope of the invention, as claimed, but is merely representative of presently preferred embodiments of the invention.

This application discloses variants of the casein kinase 1 delta and casein kinase 1 epsilon genes. These genes and the encoded proteins are highly related to each other, having over 98 percent identity in their catalytic domains, while being more divergent elsewhere. Identification of mutations in these genes in human subjects affected with FASPS implicates them in the human circadian clock. Although the casein kinase I epsilon gene has been implicated in the circadian clock of hamsters, there is no precedence for ck I delta mutations affecting the circadian clock.

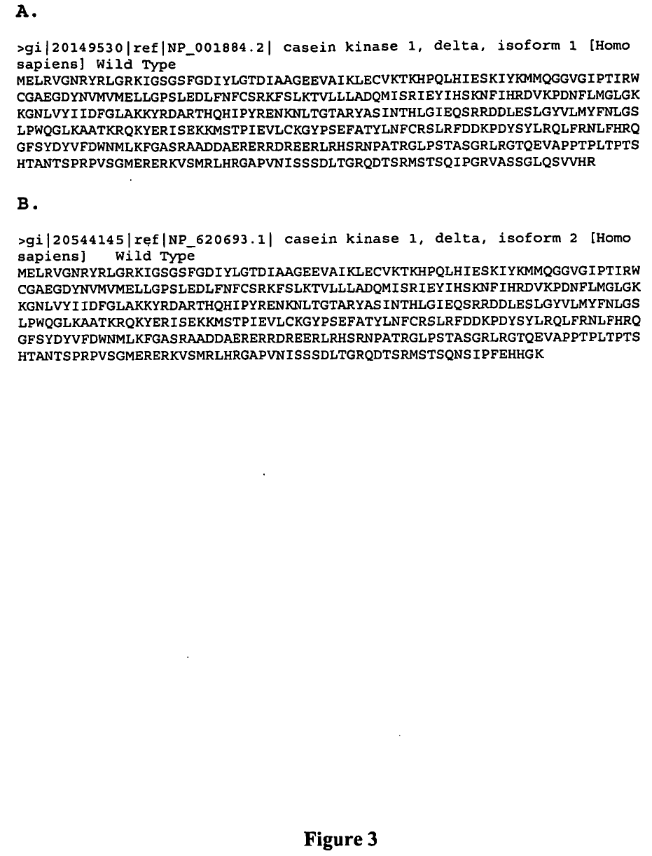

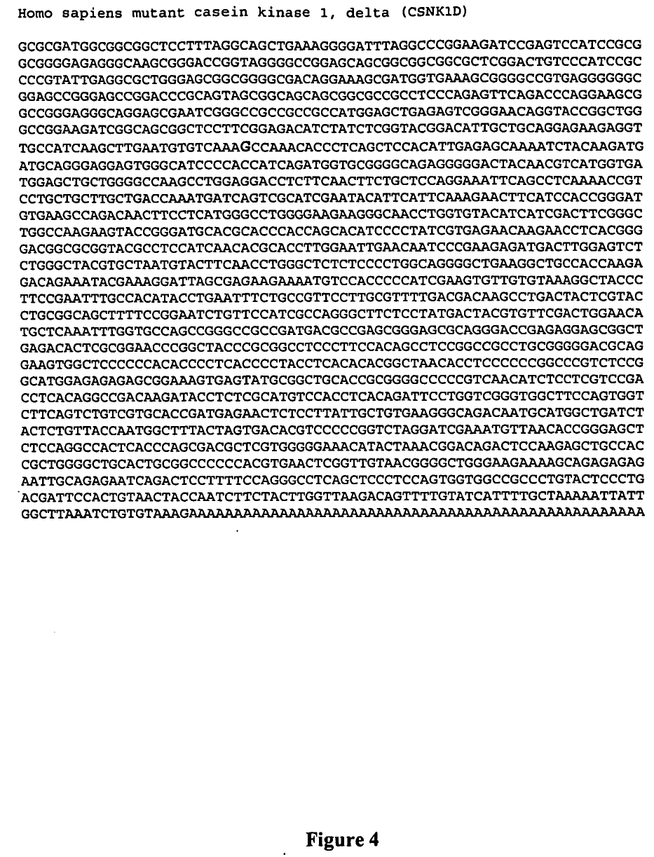

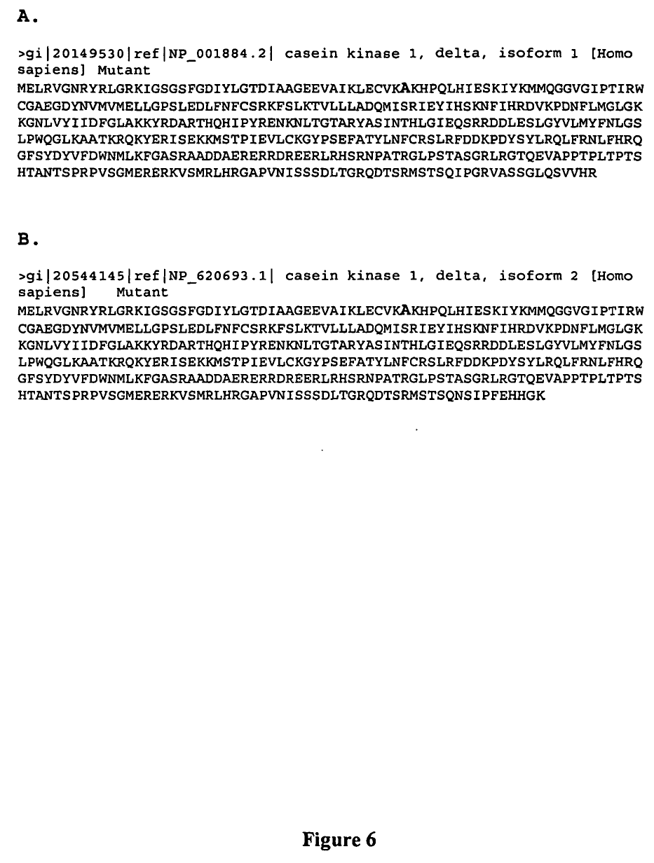

The invention first includes a variant of human casein kinase I delta isoform one encoded by the nucleic acid sequence laid out in FIG. 4 (SEQ ID NO: 5). When compared with the wild-type polynucleotide sequence encoding casein kinase I delta isoform one shown in FIG. 1 (SEQ ID NO: 1), SEQ ID NO: 5 has a mutation at the nucleotide at position 446 (shown in boldface type). This mutation specifically involves the substitution of a guanine (G) for the adenine (A) found in the wild-type nucleic acid sequence of FIG. 1 (SEQ ID NO: 1). As a result, the mutant nucleic acid of SEQ ID NO: 5 encodes mutant casein kinase I delta isoform 1 of FIG. 6A (SEQ ID NO: 7). The mutant casein kinase I delta isoform one of FIG. 6A (SEQ ID NO: 7) differs from the wild-type casein kinase I delta isoform one of FIG. 3A (SEQ ID NO: 3) in that alanine (A) is substituted for threonine (T) at position 44 in the amino acid sequence.

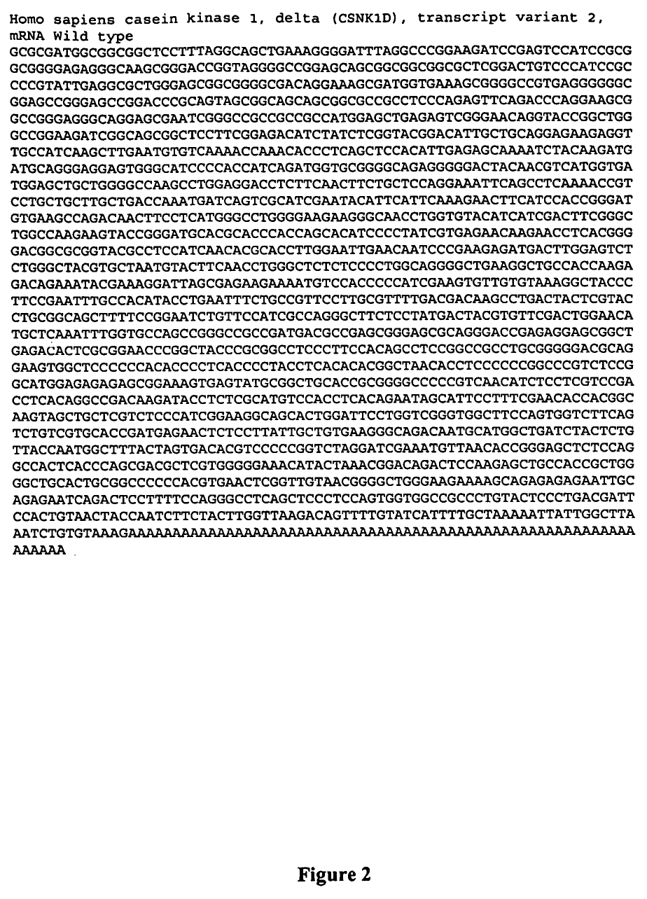

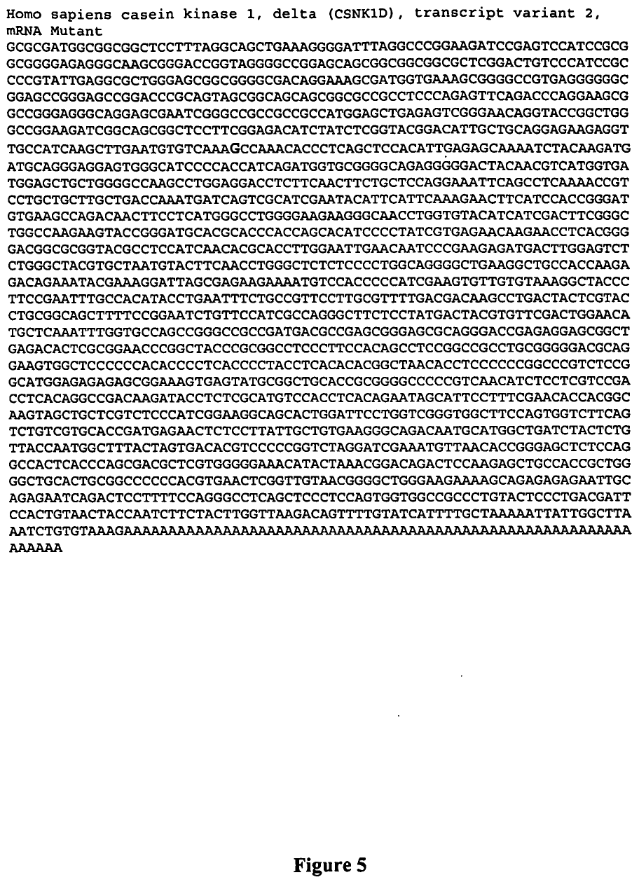

The invention next includes a variant of human casein kinase I delta isoform two encoded by the nucleic acid sequence laid out in FIG. 5 (SEQ ID NO: 6). When compared with the wild-type polynucleotide sequence encoding casein kinase I delta isoform two shown in FIG. 2 (SEQ ID NO: 2), SEQ ID NO: 6 has a mutation at the nucleotide at position 446 (shown in boldface type). This mutation specifically involves the substitution of a guanine (G) for the adenine (A) found in the wild-type nucleic acid sequence of FIG. 2 (SEQ ID NO: 2). As a result, the mutant nucleic acid of SEQ ID NO: 6 encodes mutant casein kinase I delta isoform two of FIG. 6B (SEQ ID NO: 8). The mutant casein kinase I delta isoform two of FIG. 6B (SEQ ID NO: 8) differs from the wild-type casein kinase I delta isoform two of FIG. 3B (SEQ ID NO: 4) in that alanine (A) is substituted for threonine (T) at position 44 in the amino acid sequence.

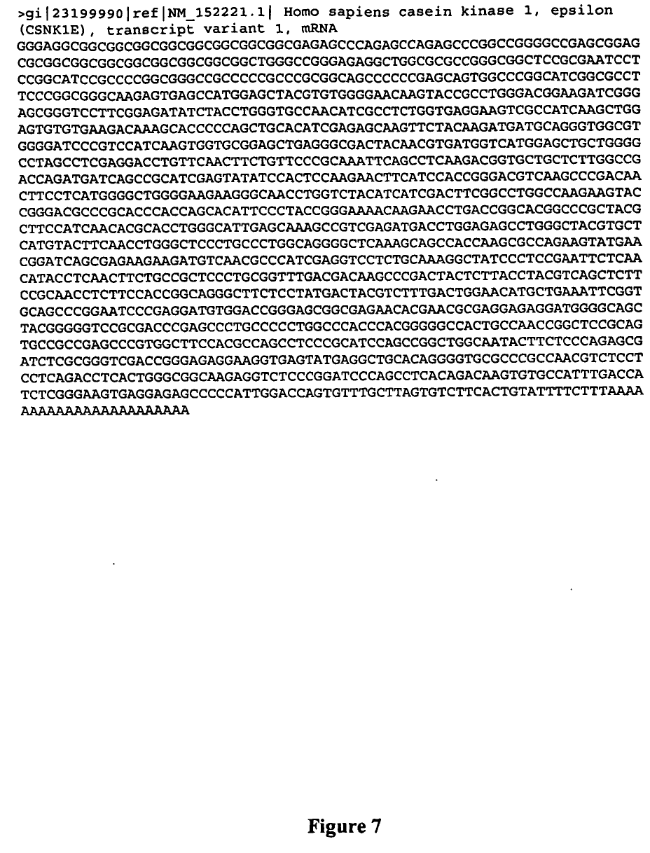

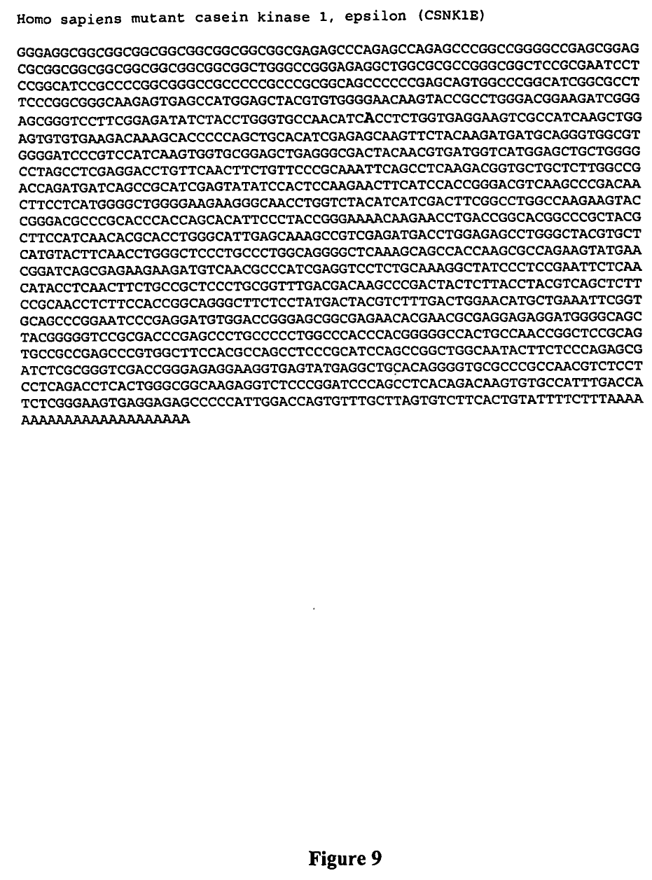

The invention next includes a variant of human casein kinase I epsilon encoded by the nucleic acid laid out in FIG. 9 (SEQ ID NO: 11). When compared with the wild-type sequence of casein kinase I epsilon shown in FIG. 7 (SEQ ID NO: 9), the mutant found in SEQ ID NO: 11 has a mutation at the nucleotide at position 320 (shown in boldface type in FIG. 9). This mutation specifically involves the substitution of an adenine (A) for a guanine (G) found in the wild-type nucleic acid of FIG. 7 (SEQ ID NO: 9).

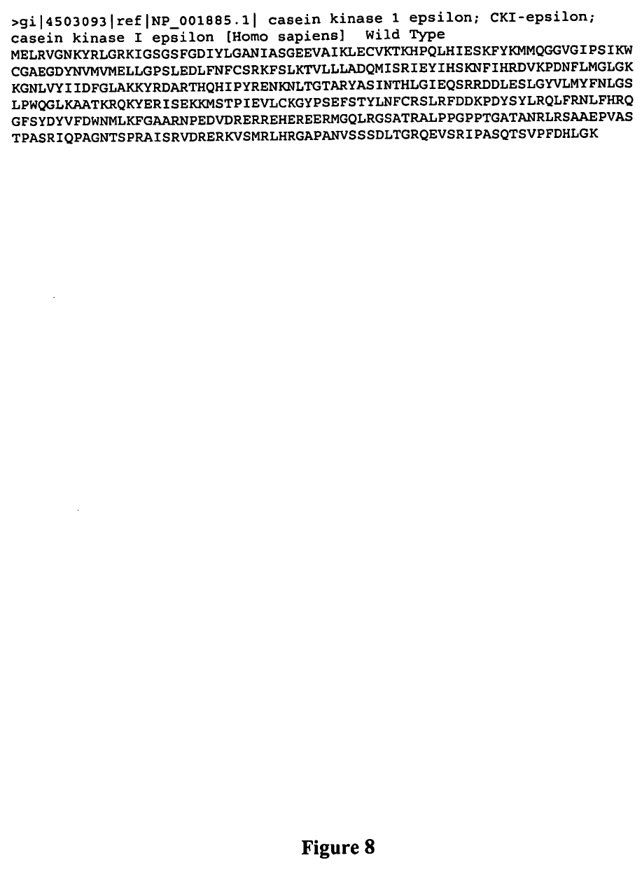

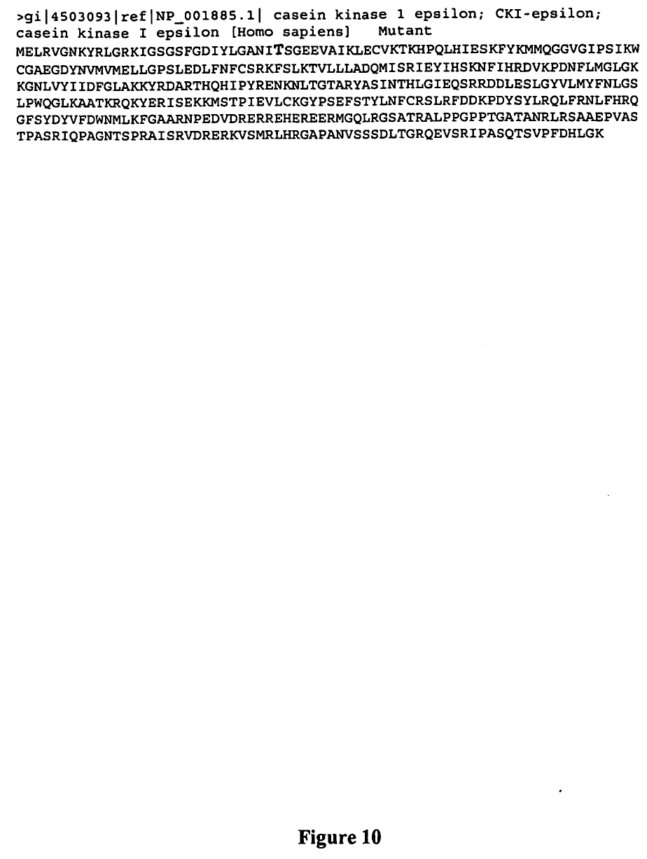

As a result, the mutant nucleic acid of SEQ ID NO: 11 encodes a mutant casein kinase I epsilon enzyme shown in FIG. 10 (SEQ ID NO: 12). This mutant enzyme includes an amino acid substitution at position 30 (shown in boldface type). This mutation specifically results in threonine (T) being substituted for an alanine (A) normally found at position 30 in the wild-type casein kinase I epsilon shown in FIG. 8 (SEQ ID NO: 10).

As claimed herein, the invention provides isolated nucleic acid sequences or polynucleotides such as those of SEQ ID NOS: 5, 6, and II, which encode mutant casein kinase I delta and epsilon mutant polypeptides shown in SEQ ID NOS: 7, 8, and 12. As used herein, the term “isolated nucleic acid” is used to denote a nucleic acid having a structure which is not identical to that of any naturally-occurring nucleic acid, or to that of any fragment of any naturally-occurring genomic nucleic acid spanning more than three separate genes. The term therefore covers, for example, (a) a DNA which has the sequence of part of a naturally-occurring genomic DNA molecule, but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally-occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such as a cDNA or genomic DNA library.

The term polynucleotides further includes polynucleotides composed of naturally-occurring nucleotides, sugars and internucleotide (or “backbone”) linkages, polynucleotides having modified nucleotides, sugars, or backbone linkages, and polynucleotides having mixed natural and modified nucleotides, sugars, and backbones or other non-naturally occurring portions that have similar function to naturally-occurring compounds.

The polynucleotides provided in the present invention may take the form of RNA including mRNA, or DNA such as cDNA and genomic DNA obtained by methods including, but not limited to cloning techniques, chemical synthetic techniques or a combination thereof. The DNA polynucleotides of the invention may be either double- or single-stranded chains. Single-stranded DNA polynucleotides according to the invention may be coding or “sense” chains or non-coding or “antisense” chains. Coding chains coding for the polypeptides of the invention may be identical to a coding sequence in the polynucleotide shown in SEQ ID NOS: 5, 6, and 11. Further, as a result of the degenerate nature of the genetic code, the polynucleotides of the invention may instead simply be polynucleotides having a different sequence from SEQ ID NOS: 5, 6, and 11, but still coding for the polypeptides of SEQ ID NOS: 7, 8, and 12.

Polynucleotides of the invention coding for the polypeptides of SEQ ID NOS: 7, 8, and 12 may include more than just regions coding for the mature polypeptide. Indeed, as is known to one of ordinary skill in the art, such coding sequences may be combined with additional coding sequences such as, but not limited to, sequences coding for a leader or a secretory sequence; coding sequences for the mature polypeptide with or without said additional sequence, plus additional non-coding sequences including non-coding 5′ and 3′ sequences and introns such as a non-translating sequences which may be transcribed, and which may play a role in mRNA processing for stability and in ribosome binding of mRNA including transcription, splicing and polyadenylation signaling. Similarly, the polynucleotides of the invention may be attached to polynucleotide coding regions for marker sequences such as peptides to facilitate purification of the resulting fusion polypeptide.

As used herein, the phrase “polynucleotide coding for the polypeptide” is used to denote, as a result of the degeneracy of the genetic code, polynucleotides containing any sequences coding for the polypeptide of this invention, particularly for mutants of human casein kinase I delta isoforms one and two and casein kinase I epsilon having the amino acid sequences shown in SEQ ID NO: 7, 8, and 12. This phrase also encompasses polynucleotides containing a single continuous region as well as polynucleotides containing multiple non-continuous regions (interrupted by sequences such as introns) and sequences with added regions may contain coding and/or non-coding regions. Further, the present invention relates to varieties of the above-described polynucleotide coding for fragments, analogues and derivatives of polypeptides having the amino acid sequences of SEQ ID NO: 7, 8, and 12. The varieties of said polynucleotides may be natural varieties such as natural allele varieties or varieties not known in the nature. Such non-natural polynucleotide varieties can be created by mutagenic techniques known to one of ordinary skill in the art.

“Complementary polynucleotides” are those capable of hybridizing with specified nucleic acid sequences of the invention. Such hybridization may take the form of Watson-Crick pairing, but may also encompass other forms of hybridization known to one of ordinary skill in the art.

The present invention also provides fragments, analogues and derivatives of the polypeptides of the invention. When the polypeptides of SEQ ID NOS: 7, 8, and 12 are referred to, the terms “fragments”, “derivatives” and “analogues” refer to polypeptides having substantially the same biological functions or activities as those of SEQ ID NOS: 7, 8, and 12. Such analogues include, among others, proproteins which can be activated by cleavage of the part of proprotein, to produce active mature polypeptides. The polypeptides of this invention may be recombinant polypeptides, natural polypeptides or synthetic polypeptides, among which recombinant polypeptides are preferable. Fragments, derivatives or analogues of the polypeptides of SEQ ID NOS: 7, 8, and 12 may be: (i) those wherein one or more amino acid residues are replaced by conserved or non-conserved amino acid residues (preferably conserved amino acids) and such replaced amino acid residues are encoded or not encoded by genetic code; (ii) those wherein one or more amino acid residues contain substituent groups; (iii) those wherein the mature polypeptide is fused to other compounds such as a compound (e.g., polyethylene glycol) increasing the half-life of the polypeptide; or (iv) those wherein additional amino acids such as a leader or secretary sequence, or a sequence used for purification of the mature polypeptide or proprotein sequences are fused to the mature polypeptide. Such fragments, derivatives and analogues are considered to be within the range of those skilled in the art on the basis of the description of this specification.

Thus, the invention provides polypeptides having the amino acid sequences shown in SEQ ID NOS: 7, 8, and 12, as well as varieties, analogues, derivatives and fragments thereof, as well as varieties, analogues and derivatives of the fragments. Some such variations may include those having conservative amino acid substitutions. In such polypeptides, amino acids are replaced by other amino acids having similar characteristics. Such replacements generally leave the polypeptide as a whole with substantially similar activity. Typical conservative amino acid replacements include mutual replacement of aliphatic amino acids Ala, Val, Leu and Ile, mutual replacement of hydroxyl residues Ser and Thr, the exchange of acidic residues Asp and Glu, replacement between amide residues Asn and Gln, exchange between basic residues Lys and Arg, and exchange between aromatic residues Phe and Tyr.

The present invention further provides vectors containing the polynucleotides of the invention, host cells genetically manipulated by such vectors, and methods of production of the polypeptides of the invention using such host cells.

The polypeptides of the invention may be expressed by genetically manipulating host cells such that the polynucleotides of the invention are integrated into their genome. In one example of this, such polynucleotide may be integrated into host cells using techniques such as infection, transduction, transfection, transvection and transformation. The polynucleotides may be integrated alone or in concert with other secondary polynucleotides. Such secondary polynucleotides may be integrated independently, or they may be integrated with, or by linking to, the polynucleotides of the invention. In one example of this, the polynucleotides of the invention may be transfected into host cells along with another polynucleotide coding for a selectable marker using a method such as co-transfection in mammalian cells and by a standard method for selection.

Alternatively, the polynucleotides of the invention may be linked to a vector construct containing a selectable marker for proliferation in a host. Such vector constructs may be integrated into specific host cells using the techniques described above. Vector constructs may be integrated as DNA in precipitates such as calcium phosphate precipitates or in the form of complexes with charged lipids into plasmid vectors. Electroporation can also be used to integrate the polynucleotides of the invention into hosts. When the vector is a virus, the virus may be packaged in vitro or integrated in package cells and then the packaged virus may be used to transduce cells. A large variety of techniques suitable for integrating polynucleotides of the invention into cells are well-known to those skilled in the art.

According to the invention, the vector may be a plasmid vector, a single- or double-stranded phage vector or a single- or double-stranded RNA or DNA virus vector. Such a vector can be integrated as a polynucleotide, preferably DNA into cells by any known techniques for integrating DNA and RNA into cells. In the case of the phage and virus vectors, the vector may be integrated preferably as a package or capsulated virus by well-known techniques for infection and transduction. The virus vector may be a replicating component, or it may alternately be deficient in replication.

Vectors of the invention drive expression of the polypeptides of the present invention. Generally, such vectors consist of a cis-acting regulatory region which is effective for expression in host cells and linked in an operable manner to the polynucleotide to be expressed. A suitable trans-acting factor is supplied by a host, by a complementing vector or by a vector itself after being integrated into a host. In an example preferable in this respect, the vector is subjected to specific expression. Such specific expression can be inducible expression or expression in only a certain type of cells and cell-specific expression or both inducible and cell-specific expression. Some vectors may be inducible by easily-modified environmental factors such as temperature and nutritive additives. Various vectors suitable for this mode of this invention, including constitutional and inducible expression vectors for use in prokaryotic and eukaryotic hosts, are well known to those skilled in the art.

Host cells of the invention manipulated to express the polypeptides of the invention may be cultured in a usual nutrient medium, or the medium may be modified to activate a promoter, to select a transformant, or to amplify the gene. Various expression vectors can be used for expression of the polypeptide of this invention. Such vectors include chromosomes, episomes and viral inducible vectors, e.g. microbial plasmids, bacteriophages, yeast episomes, vectors derived from yeast chromosome elements, viruses such as baculovirus, papovavirus, SV40, vaccinia virus, adenovirus, fowipox virus, pseudo-rabies virus and retrovirus, vectors derived from plasmids and genetic elements in bacteriophages, and vectors such as cosmids and phagimides, derived from a combination of the above. In general, any vectors suitable for maintaining the polynucleotide, multiplying and expressing it to produce the polypeptides of the invention may be used.

DNA polynucleotide sequences of the invention may be inserted into a suitable vector using a wide variety of procedures. In general, a DNA sequence for expression and an expression vector are first cleaved with one or more restriction endonuleases, and the resulting restriction fragments are ligated by T4 DNA ligase whereby the DNA sequence is ligated to the expression vector. The procedure for restriction and ligation used for this purpose is generally known to those skilled in the art. The DNA sequence in the expression vector is coupled to a suitable expression regulatory sequence containing elements such as a promoter for directing mRNA translation to render the expression vector operable. Typical examples of such promoters include .lambda.-phage PL promoter, E. coli lac, trp and tac promoters, SV40 early and late promoters and retrovirus LTR. A large number of other promoters useful in the present invention are well-known and can be ordinarily used by those skilled in the art.

Suitable DNA polynucleotides are introduced into a suitable host by use of a wide variety of well-known methods suitable for expressing the desired polypeptides. Typical examples of suitable hosts include microbial cells such as E. coli, Streptomyces and Salmonella typhimurium cells; eukaryotic cells such as yeast cells; insect cells such as Drosophila S2 and SHIRONAYOTO Sf9 cells; animal cells such as CHO, COS or Bowes melanoma cells; and plant cells. Various hosts for the expression construct are well-known and can be selected ordinarily by those skilled in the art for expression of the polypeptide according to the mode of this invention in this specification.

The invention further provides fragments of nucleotides encoding portions of mutant casein kinase I delta and epsilon. More specifically, the invention provides fragments of at least 15 contiguous nucleotides of the mutant nucleic acid sequences of the invention which encompass the site of the mutation. Such fragments derive utility from their ability to selectively hybridize to the polynucleotides of the invention. Thus, the invention provides fragments of the polynucleotide of SEQ ID NO: 5 encoding the mutant of human casein kinase I delta isoform one. Such fragments include those comprising the mutated nucleotide at position 446. The invention further provides fragments of the polynucleotide of SEQ ID NO: 6 encoding the mutant of human casein kinase I delta isoform two. Such fragments include those comprising the mutated nucleotide at position 446. Still further, the invention provides fragments of the polynucleotide of SEQ ID NO: 11 encoding the mutant of human casein kinase I epsilon. Such fragments include those comprising the mutated nucleotide at position 320.

Hybridizing nucleic acids of this type may be used, for example, as cloning probes, primers (such as PCR primers), or as diagnostic probes. Hybridization of such a probe to a nucleic acid sample is generally performed under stringent conditions. Nucleic acid duplex or hybrid stability is expressed as the melting temperature or Tm, which is the temperature at which a probe dissociates from a target DNA. This melting temperature is used to define the required stringency of hybridization conditions. If sequences are to be identified that are related to and/or substantially identical to the probe, rather than identical to the probe, then it is useful to first establish the lowest temperature at which only homologous hybridization occurs with a particular concentration of salt.

Assuming that 1% mismatching results in a 1° C. decrease in the Tm, the temperature of the final wash in a hybridization reaction is reduced accordingly. In an example, if sequences having greater than 95% identity with the probe are sought, the final wash temperature should be decreased by 5° C. In practice, the change in Tm may vary between 0.5° C. and 1.5° C. per 1% mismatch. Stringent conditions involve hybridizing at 68° C. in 5×SSC/5× Denhardt's solution/1.0% SDS, and washing in 0.2×SSC/0.1% SDS at room temperature. Moderately stringent conditions include washing in 3×SSC at 42° C. The parameters of salt concentration and temperature may be varied to achieve the optimal level of identity between the probe and a target nucleic acid. Additional guidance regarding hybridization conditions is available to one of skill in the art in a reference such as, but not limited to, Sambrook et al., mentioned above.

The invention also provides methods of screening for potential agents that regulate the mutants of casein kinase I epsilon and casein kinase I delta that are identified herein. In a first such method, a cell expressing or overexpressing the polypeptide of SEQ ID NO: 7, mutant casein kinase I delta, isoform 1, or a fragment thereof, is contacted with a test compound. Following contact, the activity of the polypeptide of SEQ ID NO: 7 is measured. Those compounds that either increase or decrease the activity of the polypeptide of SEQ ID NO: 7 are considererd potential agents that regulate the activity of mutant casein kinase I delta, isoform 1.

In a next method, a cell expressing or overexpressing the polypeptide of SEQ ID NO: 8, mutant casein kinase I delta, isoform 2, or a fragment thereof, is contacted with a test compound. Following contact, the activity of the polypeptide of SEQ ID NO: 8 is measured. Those compounds that either increase or decrease the activity of the polypeptide of SEQ ID NO: 7 are considered potential agents that regulate the activity of mutant casein kinase 1 delta, isoform 2.

In a further method, a cell expressing or overexpressing the polypeptide of SEQ ID NO: 12, mutant casein kinase I epsilon, or a fragment thereof, is contacted with a test compound. Following contact, the activity of the polypeptide of SEQ ID NO: 12 is measured. Those compounds that either increase or decrease the activity of mutant casein kinase I epsilon are considered potential agents that regulate the activity of the mutant casein kinase 1 epsilon polypeptide.

In those methods in which the cells used express a fragment of SEQ ID NOS: 7 and 8, according to the methods of the invention the fragment would include position 44 of the mutant polypeptide. In those methods in which the cells used express a fragment of SEQ ID NO: 12, according to the methods of the invention, the fragment would include position 30 of the mutant polypeptide.

The potential agents for regulating the activity of mutant casein kinase I epsilon and delta of the invention, as well as the polypeptides and polynucleotides of the invention may be combined with pharmaceutically acceptable carriers to provide pharmaceutical compositions for treating biological conditions or disorders such as those briefly noted herein in organisms such as human patients. The particular carrier employed in these pharmaceutical compositions may take a wide variety of forms depending upon the type of administration desired, e.g., intravenous, oral, topical, suppository or parenteral. In some configurations of the invention, the polynucleotides of the invention may be utilized in a chemically-modified form, or with carriers such as the copolymers taught in U.S. Pat. No. 6,586,180.

In preparing the compositions in oral liquid dosage forms (e.g., suspensions, elixirs and solutions), typical pharmaceutical media, such as water, glycols, oils, alcohols, flavoring agents, preservatives, coloring agents and the like can be employed. Similarly, when preparing oral solid dosage forms (e.g., powders, tablets and capsules), carriers such as starches, sugars, diluents, granulating agents, lubricants, binders, disintegrating agents and the like will be employed. Due to their ease of administration, tablets and capsules represent the most advantageous oral dosage form for the pharmaceutical compositions of the present invention.

For parenteral administration, the carrier may typically comprise sterile water, although other ingredients that aid in solubility or serve as preservatives may also be included. Furthermore, injectable suspensions may also be prepared, in which case appropriate liquid carriers, suspending agents and the like will be employed.

For topical administration, the compounds of the present invention may be formulated using bland, moisturizing bases, such as ointments or creams. Examples of suitable ointment bases are petrolatum, petrolatum plus volatile silicones, lanolin, and water in oil emulsions.

As recognized by those skilled in the art, the particular quantity of pharmaceutical composition according to the present invention administered to a patient will depend upon a number of factors, including, without limitation, the biological activity desired, the condition of the patient, and tolerance for the drug.

The casein kinase I delta and casein kinase I epsilon genes and the proteins which they encode of the invention are likely excellent potential targets for the development of therapeutic agents that could alter human sleep. As enzymes, casein kinases are conducive to manipulations by chemicals that can increase or decrease their activity. The identification of genetic variants in these genes and their implication in familial advanced sleep phase syndrome provides the rationale for developing such agents and using them in efforts to modulate human sleep.

1. An isolated nucleic acid molecule comprising a polynucleotide selected from the group consisting of:
(a) a polynucleotide coding for a polypeptide consisting of the amino acid sequence of SEQ ID NO: 7;
(b) a degenerate polynucleotide coding for a polypeptide coding for the amino acid sequence of SEQ ID NO: 7;
(c) a polynucleotide complementary to the polynucleotide of (a) or (b); and
(d) a polynucleotide consisting of at least 15 contiguous nucleotides from the polynucleotide of (a), (b), or (c) including nucleotide 446.

2. The polynucleotide of claim 1, wherein the polynucleotide is DNA.

3. The polynucleotide of claim 1, wherein the polynucleotide is RNA.

4. The polynucleotide of claim 3, wherein the polynucleotide comprises SEQ ID NO: 5.

5. A vector comprising the DNA polynucleotide of claim 2.

6. An isolated host cell comprising the vector of claim 5.

7. A method of producing a polypeptide, comprising the steps of allowing the host cell of claim 6 to express a polypeptide encoded by the polynucleotide.

8. A method of producing cells expressing a polypeptide comprising the steps of transforming or transfecting cells with the vector of claim 5 and allowing the cells to express the polypeptide encoded by the polynucleotide of the vector.

9. An isolated polypeptide comprising the amino acid sequence of SEQ ID NO: 7.

10. A method of screening for potential agents that regulate the activity of the polypeptide of SEQ ID NO: 7 in a cell comprising the steps of:
contacting a cell with a test compound, wherein the cell expresses or overexpresses the polypeptide of SEQ ID NO: 7 or a fragment thereof including position 44;
measuring the level of activity of the polypeptide of SEQ ID NO: 7 in the cell, wherein a test compound which increases or decreases the activity of the polypeptide of SEQ ID NO: 7 or fragment thereof in the cell is a potential agent that regulates the activity of the polypeptide of SEQ ID NO: 7.

11. An isolated nucleic acid molecule comprising a polynucleotide selected from the group consisting of:
(a) a polynucleotide coding for a polypeptide consisting of the amino acid sequence of SEQ ID NO: 8;
(b) a degenerate polynucleotide coding for a polypeptide coding for the amino acid sequence of SEQ ID NO: 8;
(c) a polynucleotide complementary to the polynucleotide of (a) or (b); and
(d) a polynucleotide consisting of at least 15 contiguous nucleotides from the polynucleotide of (a), (b), or (c) including nucleotide 446.

12. The polynucleotide of claim 11, wherein the polynucleotide is DNA.

13. The polynucleotide of claim 11, wherein the polynucleotide is RNA.

14. The polynucleotide of claim 13, wherein the polynucleotide comprises SEQ ID NO: 6.

15. A vector comprising the DNA polynucleotide of claim 12.

16. An isolated host cell comprising the vector of claim 15.

17. A method of producing a polypeptide, comprising the steps of allowing the host cell of claim 16 to express a polypeptide encoded by the polynucleotide.

18. A method of producing cells expressing a polypeptide comprising the steps of transforming or transfecting cells with the vector of claim 15 and allowing the cells to express the polypeptide encoded by the polynucleotide of the vector.

19. An isolated polypeptide comprising the amino acid sequence of SEQ ID NO: 8.

20. A method of screening for potential agents that regulate the activity of the polypeptide of SEQ ID NO: 8 in a cell comprising the steps of:
contacting a cell with a test compound, wherein the cell expresses or overexpresses the polypeptide of SEQ ID NO: 8 or a fragment thereof including position 44;
measuring the level of activity of the polypeptide of SEQ ID NO: 8 in the cell, wherein a test compound which increases or decreases the activity of the polypeptide of SEQ ID NO: 8 or fragment thereof in the cell is a potential agent that regulates the activity of the polypeptide of SEQ ID NO: 8.

21. An isolated nucleic acid molecule comprising a polynucleotide selected from the group consisting of:
(a) a polynucleotide coding for a polypeptide consisting of the amino acid sequence of SEQ ID NO: 12;
(b) a degenerate polynucleotide coding for a polypeptide coding for the amino acid sequence of SEQ ID NO: 12;
(c) a polynucleotide complementary to the polynucleotide of (a) or (b); and
(d) a polynucleotide consisting of at least 15 contiguous nucleotides from the polynucleotide of (a), (b), or (c) including nucleotide 320.

22. The polynucleotide of claim 21, wherein the polynucleotide is DNA.

23. The polynucleotide of claim 21, wherein the polynucleotide is RNA.

24. The polynucleotide of claim 23, wherein the polynucleotide comprises SEQ ID NO: 11.

25. A vector comprising the DNA polynucleotide of claim 22.

26. An isolated host cell comprising the vector of claim 21.

27. A method of producing a polypeptide, comprising the steps of allowing the host cell of claim 26 to express a polypeptide encoded by the polynucleotide.

28. A method of producing cells expressing a polypeptide comprising the steps of transforming or transfecting cells with the vector of claim 25 and allowing the cells to express the polypeptide encoded by the polynucleotide of the vector.

29. An isolated polypeptide comprising the amino acid sequence of SEQ ID NO: 12.

30. A method of screening for potential agents that regulate the activity of the polypeptide of SEQ ID NO: 12 in a cell comprising the steps of:
contacting a cell with a test compound, wherein the cell expresses or overexpresses the polypeptide of SEQ ID NO: 12 or a fragment thereof including position 30;
measuring the level of activity of the polypeptide of SEQ ID NO: 12 in the cell, wherein a test compound which increases or decreases the activity of the polypeptide of SEQ ID NO: 12 or fragment thereof in the cell is a potential agent that regulates the activity of the polypeptide of SEQ ID NO: 12.
